(19) United States
(12) Patent Application Publication
(10) Pub. No.: US 2026/0242344 A1
(43) Pub. Date: Aug. 20, 2026

(54) A PROCESS FOR PREPARING HYDROXYPHENYL-1,3,5-TRIAZINE COMPOUNDS

(71) Applicant: BASF SE, Ludwigshafen am Rhein (DE)

(72) Inventors: Abhijeet ROY, Navi Mumbai (IN); Mushtaq PATEL, Navi Mumbai (IN); Mileen KADAM, Navi Mumbai (IN); Shivnath HARPAD, Mumbai (IN); Shilpa KORDE, Navi Mumbai (IN); Ramraj BHATTA, Thane (IN)

(21) Appl. No.: 18/876,764

(22) PCT Filed: Jun. 20, 2023
(86) PCT No.: PCT/EP2023/066610

§ 371 Date: Dec. 19, 2024

(30) Foreign Application Priority Data
Jun. 22, 2022 (EP) 22180418.0

(51) Int. Cl. C07D 251/24

(52) U.S. Cl. CPC C07D 251/24

(57) ABSTRACT

The presently claimed invention relates to a process for preparing hydroxyphenyl-1,3,5-triazine compounds. Particularly, the presently claimed invention relates to a process for preparing hydroxyphenyl-1,3,5-triazine compounds by reaction of corresponding 2-chloro-1,3,5-triazine compound with a phenol. The process comprises the steps of preparing a deep eutectic mixture (DEM) by mixing at least one quaternary ammonium salt and at least one phenol, and reacting at least one substituted 2-chloro-1,3,5-triazine compound and the deep eutectic mixture to obtain the hydroxy-phenyl-1,3,5-triazine compound of general formula (I).

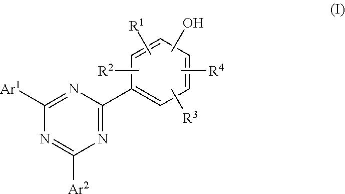

### FIELD OF THE INVENTION

The presently claimed invention relates to a process for preparing hydroxyphenyl-1,3,5-triazine compounds. Particularly, the presently claimed invention relates to a process for preparing hydroxyphenyl-1,3,5-triazine compounds by reaction of the corresponding 2-chloro-1,3,5-triazine compound and a phenol.

### BACKGROUND OF THE INVENTION

Hydroxyphenyl-1,3,5-triazines form a class of commercially useful compounds or intermediates. Compounds such as Tinuvin-477®, Tinuvin 460®, and Tinosorb-S® are used as UV-filters and their preparation involves hydroxyphenyl-1,3,5-triazines intermediates.

The commercial preparation of hydroxyphenyl-1,3,5-triazine compounds involves Friedel-Crafts alkylation of corresponding 2-chloro-1,3,5-triazine compound with a phenol.

CN105218466 describes a method for preparing 2,4,6-tri(2′,4′-dihydroxyphenyl)-1,3,5-triazine involving reaction of cyanuric chloride and resorcinol in an aprotic or protic solvent in the presence of a metal oxide or supported solid acid catalyst.

However, the known processes for preparation of hydroxyphenyl-1,3,5-triazine compounds are associated with drawbacks such as use of a metal catalyst, which may be toxic. The post reaction work-up procedures are tedious and generate hazardous effluent. Often the triazine starting material has a low solubility, thereby requiring the use of a harmful solvent.

Accordingly, it is an object of the presently claimed invention to provide a process for the preparation of hydroxyphenyl-1,3,5-triazine compounds that overcomes one or more of the above-mentioned drawbacks. It is an object of the presently claimed invention to provide a process involving a non-metal catalyst for the reaction. Further, it is an object of the presently claimed invention to provide a process involving an easy work-up procedure.

### SUMMARY OF THE INVENTION

It was surprisingly found that the presently claimed invention provides a process for the preparation of hydroxyphenyl-1,3,5-triazine compounds that overcomes one or more of the abovementioned drawbacks. Particularly, the Friedel-Crafts alkylation of corresponding 2-chloro-1,3,5-triazine compound and a phenol is carried out in the presence of a non-metal catalyst and the work-up procedure is simple.

Accordingly, one aspect of the presently claimed invention is a process for preparing a hydroxyphenyl-1,3,5-triazine compound of general formula (I)

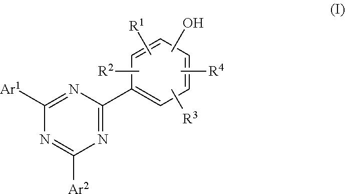

wherein
Ar¹ and Ar² are identical or different and independently selected from substituted or unsubstituted C₆-C₁₈ aryl, or a moiety of the formula (A),

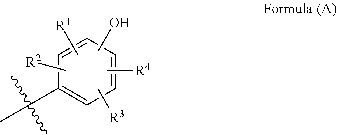

wherein
R¹ to R⁴ are independently selected from hydrogen, —OH, —CN, —COOH, substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, substituted or unsubstituted C₇-C₂₄ arylalkyl, —OR⁵, —COOR⁵, —NR⁵R⁶, or —C(═O)NR⁵R⁶, wherein R⁵ is a linear or branched, substituted or unsubstituted C₁-C₁₈ alkyl and R⁶ is a linear or branched, substituted or unsubstituted C₁-C₁₈ alkyl.

The process comprises the steps of
a) preparing a deep eutectic mixture (DEM) (IV) by mixing at least one quaternary ammonium salt of general formula (II) and at least one phenol of general formula (III),

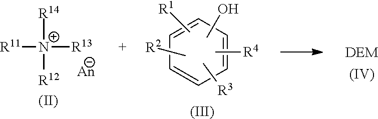

wherein R¹¹ to R¹⁴ are independently selected from linear or branched, substituted or unsubstituted C₁-C₁₈ alkyl, or substituted or unsubstituted C₇-C₂₄ arylalkyl, or
R¹¹ and R¹² together with the nitrogen atom to which they are bonded form a substituted or unsubstituted, saturated or unsaturated 5- to 12-membered cycloalkyl ring that optionally contains 1, 2 or 3 heteroatom(s) selected from O, N, or S as ring member(s); and An⁻ is selected from chloride, bromide, iodide, fluoride, sulfate, sulfonate, alkylsulfate, or carboxylate, and R¹ to R⁴ as defined above; and
b) reacting at least one substituted 2-chloro-1,3,5-triazine compound of general formula (V) and the deep eutectic mixture obtained according to step (a) to obtain the hydroxyphenyl-1,3,5-triazine compound of general formula (I),

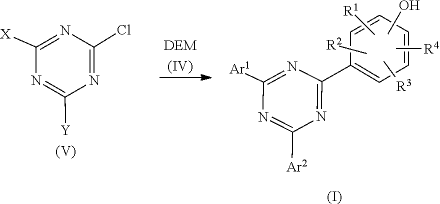

wherein
X is chloro or Ar¹, and Y is chloro or Ar².

### DETAILED DESCRIPTION OF THE INVENTION

Before the present compositions and formulations of the presently claimed invention are described, it is to be understood that this invention is not limited to particular compositions and formulations described, since such compositions and formulation may, of course, vary. It is also to be understood that the terminology used herein is not intended to be limiting, since the scope of the presently claimed invention will be limited only by the appended claims.

If hereinafter a group is defined to comprise at least a certain number of embodiments, this is meant to also encompass a group which preferably consists of these embodiments only. Furthermore, the terms ‘first’, ‘second’, ‘third’ or ‘a’, ‘b’, ‘c’, etc. and the like in the description and in the claims, are used for distinguishing between similar elements and not necessarily for describing a sequential or chronological order. It is to be understood that the terms so used are interchangeable under appropriate circumstances and that the embodiments of the presently claimed invention described herein are capable of operation in other sequences than described or illustrated herein. In case the terms ‘first’, ‘second’, ‘third’ or ‘(A)’, ‘(B)’ and ‘(C)’ or ‘(a)’, ‘(b)’, ‘(c)’, ‘(d)’, ‘i’, ‘ii’ etc. relate to steps of a method or use or assay there is no time or time interval coherence between the steps, that is, the steps may be carried out simultaneously or there may be time intervals of seconds, minutes, hours, days, weeks, months or even years between such steps, unless otherwise indicated in the application as set forth herein above or below.

Furthermore, the ranges defined throughout the specification include the end values as well i.e. a range of 1 to 10 implies that both 1 and 10 are included in the range. For the avoidance of doubt, applicant shall be entitled to any equivalents according to applicable law.

In the following passages, different aspects of the presently claimed invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to ‘one embodiment’ or ‘an embodiment’ means that a particular feature, structure, or characteristic described in connection with the embodiment is included in at least one embodiment of the presently claimed invention. Thus, appearances of the phrases ‘in one embodiment’ or ‘in an embodiment’ in various places throughout this specification are not necessarily all referring to the same embodiment but may refer to so.

Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the presently claimed invention, and form different embodiments, as would be understood by those in the art. For example, in the appended claims, any of the claimed embodiments can be used in any combination.

The presently claimed invention uses a deep eutectic mixture (DEM) (IV) obtained by mixing at least one quaternary ammonium salt of general formula (II) and at least one phenol of general formula (III) for conducting the Friedel-Crafts alkylation of 2-chloro-1,3,5-triazine compound and the phenol. The DEM acts as a non-metal catalyst for the Friedel-Crafts alkylation. Further, the DEM acts as a solvent for the 2-chloro-1,3,5-triazine compound, which has a low solubility. The DEM are characterized by a low vapor pressure, and they are non-flammable and are environmentally friendly solvents. Furthermore, the phenol present in the DEM acts as a reactant. The work-up procedure is simple.

Accordingly, an aspect of the presently claimed invention is directed to a process for preparing a hydroxyphenyl-1,3,5-triazine compound of general formula (I)

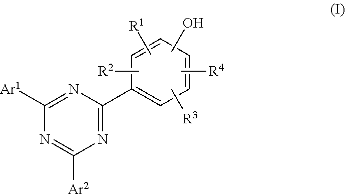

wherein
Ar¹ and Ar² are identical or different and independently selected from substituted or unsubstituted C₆-C₁₈ aryl, or a moiety of the formula (A),

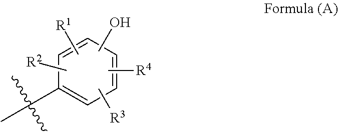

wherein
R¹ to R⁴ are independently selected from hydrogen, —OH, —CN, —COOH, substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, substituted or unsubstituted C₇-C₂₄ arylalkyl, —OR⁵, —COOR⁵, —NR⁵R⁶, or —C(═O)NR⁵R⁶, wherein R⁵ is a linear or branched, substituted or unsubstituted C₁-C₁₈ alkyl, and R⁶ is a linear or branched, substituted or unsubstituted C₁-C₁₈ alkyl.

The process comprises the steps of
a) preparing a deep eutectic mixture (DEM) (IV) by mixing at least one quaternary ammonium salt of general formula (II) and at least one phenol of general formula (III),

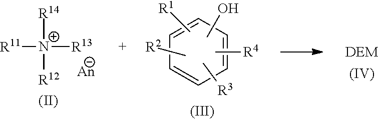

wherein R¹¹ to R¹⁴ are independently selected from linear or branched, substituted or unsubstituted C₁-C₁₈ alkyl, or substituted or unsubstituted C₇-C₂₄ arylalkyl, or
   R¹¹ and R¹² together with the nitrogen atom to which they are bonded form a substituted or unsubstituted, saturated or unsaturated 5- to 12-membered cycloalkyl ring that optionally contains 1, 2 or 3 heteroatom(s) selected from O, N, or S as ring member(s); and An⁻ is selected from chloride, bromide, iodide, fluoride, sulfate, sulfonate, alkylsulfate, or carboxylate, and R¹ to R⁴ as defined above; and
b) reacting at least one substituted 2-chloro-1,3,5-triazine compound of general formula (V) and the deep eutectic mixture obtained according to step (a) to obtain the hydroxyphenyl-1,3,5-triazine compound of general formula (I),

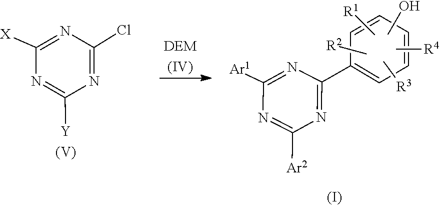

wherein
X is chloro or Ar¹, and Y is chloro or Ar².

Within the context of the presently claimed invention, the term alkyl, as used herein, refers to an acylic saturated aliphatic groups, including linear alkyl saturated hydrocarbon radical denoted by a general formula CₙH₂ₙ₊₁ and wherein n is the number of carbon atoms 1, 2, 3, 4 etc. Preferably, the alkyl refers to linear unsubstituted C₁ to C₂₄ carbon atoms, selected from methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, 1-methyl ethyl, 1-methyl propyl, 2-methyl propyl, 1-methyl butyl, 2-methyl butyl, 3-methyl butyl, 1-methyl pentyl, 2-methyl pentyl, 3-methyl pentyl, 4-methyl pentyl, 1-ethyl propyl, 1-ethyl butyl, or 2-ethyl butyl.

Within the context of the presently claimed invention, the term alkenyl, as used herein, refers to an acylic unsaturated aliphatic groups having at least one double bond, including linear alkenyl unsaturated hydrocarbon radical denoted by a general formula CₙH₂ₙ₋₁ and wherein n is the number of carbon atoms 1, 2, 3, 4 etc. Preferably, the alkenyl refers to linear unsubstituted C₂ to C₁₂ carbon atoms, selected from prop-1-enyl, prop-2-enyl, but-1-enyl, but-2-enyl, but-3-enyl, pent-1-enyl, pent-2-enyl, pent-3-enyl, pent-4-enyl, hex-1-enyl, hex-2-enyl, hex-3-enyl, hex-4-enyl, hex-5-enyl, hept-1-enyl, hept-2-enyl, hept-3-enyl, hept-4-enyl, hept-5-enyl, hept-6-enyl, oct-1-enyl, oct-2-enyl, oct-3-enyl, oct-4-enyl, oct-5-enyl, oct-6-enyl, oct-7-enyl, non-1-enyl, non-2-enyl, non-3-enyl, non-4-enyl, non-5-enyl, non-6-enyl, non-7-enyl, non-8-enyl, dec-1-enyl, dec-2-enyl, dec-3-enyl, dec-4-enyl, dec-5-enyl, dec-6-enyl, dec-7-enyl, dec-8-enyl, dec-9-enyl, undec-1-enyl, undec-2-enyl, undec-3-enyl, undec-4-enyl, undec-5-enyl, undec-6-enyl, undec-7-enyl, undec-8-enyl, undec-9-enyl, undec-10-enyl, dodec-1-enyl, dodec-2-enyl, dodec-3-enyl, dodec-4-enyl, dodec-5-enyl, dodec-6-enyl, dodec-7-enyl, dodec-8-enyl, dodec-9-enyl, dodec-10-enyl, or dodec-11-enyl.

Within the context of the presently claimed invention, the term cycloalkyl, as used herein, refers to a monocyclic and a bicyclic 3 to 24 membered saturated cycloaliphatic groups, including branched cycloalkyl saturated hydrocarbon. Preferably, the cycloalkyl refers to C₅ to C₁₀ carbon atoms, selected from cyclopentyl, cyclohexyl, cycloheptyl, cyclooctyl, cyclononyl, or cyclodecyl.

Within the context of the presently claimed invention, the term cycloalkenyl, as used herein, refers to a monocyclic and a bicyclic 3 to 10 membered unsaturated cycloaliphatic groups having at least one double bond. Preferably, the cycloalkenyl refers to C₅ to C₁₀ carbon atoms, selected from cyclopent-1-enyl, cyclopent-2-enyl, cyclopent-3-enyl, cyclohex-1-enyl, cyclohex-2-enyl, cyclohex-3-enyl, cyclohex-4-enyl, cyclohept-1-enyl, cyclohept-2-enyl, cyclohept-3-enyl, cyclohept-4-enyl, cyclohept-5-enyl, cyclooct-1-enyl, cyclooct-2-enyl, cyclooct-3-enyl, cyclooct-4-enyl, cyclooct-5-enyl, cyclooct-6-enyl, cyclonon-1-enyl, cyclonon-2-enyl, cyclonon-3-enyl, cyclonon-4-enyl, cyclonon-5-enyl, cyclonon-6-enyl, cyclonon-7-enyl, cyclodec-1-enyl, cyclodec-2-enyl, cyclodec-3-enyl, cyclodec-4-enyl, cyclodec-5-enyl, cyclodec-6-enyl, cyclodec-7-enyl, or cyclodec-8-enyl.

Within the context of the presently claimed invention, the term aryl, as used herein, refers to phenyl or naphthyl, preferably phenyl. The term substituted aryl refers to an aryl radical wherein a part or all the hydrogen atoms are replaced by substituent/s, preferably the substituents are selected from hydroxy, halogen, cyano, C₁-C₄-alkyl, or C₁-C₄-alkoxy. Preferably aryl is unsubstituted or carries 1, 2 or 3 substituents.

Within the context of the presently claimed invention, the term arylalkyl, as used herein, refers to aryl group such as phenyl or naphthyl, with an alkyl group. Preferably, arylalkyl refers to C₇-C₂₄ selected from benzyl, phenylethyl, or 2-phenylethyl. The term substituted arylalkyl refers to an aryl radical wherein a part or all the hydrogen atoms are replaced by substituent/s, preferably the substituents are selected from hydroxy, halogen, cyano, C₁-C₄-alkyl, or C₁-C₄-alkoxy. Preferably aryl is unsubstituted or carries 1, 2 or 3 substituents.

Within the context of the presently claimed invention, the term substituted group refers to a radical, wherein a part or all the hydrogen atoms are replaced by substituent/s, preferably the substituents are selected from hydroxy, halogen, cyano, C₁-C₄-alkyl, or C₁-C₄-alkoxy. The group can be an alkyl, alkenyl, cycloalkyl, cycloalkenyl, aryl, arylalkyl or the like.

Within the context of the presently claimed invention, the term deep eutectic mixture (DEM), as used herein, refers to an emerging class of mixtures characterized by significant depressions in melting points compared to those of the neat constituent components.

In a preferred embodiment, the presently claimed invention is directed to a process for preparing a hydroxyphenyl-1,3,5-triazine compound of general formula (I)

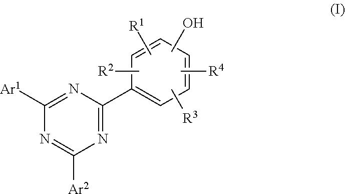

wherein
Ar¹ and Ar² are identical or different and independently selected from substituted or unsubstituted C₆-C₁₈ aryl, or a moiety of the formula (A),

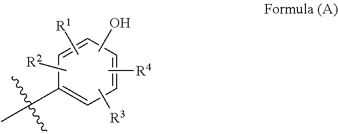

wherein
R¹ to R⁴ are independently selected from hydrogen, —OH, substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, substituted or unsubstituted C₇-C₂₄ arylalkyl, —OR⁵, or —COOR⁵, wherein R⁵ is a linear or branched, substituted or unsubstituted C₁-C₁₈ alkyl.

In a preferred embodiment, the presently claimed invention is directed to a process for preparing a hydroxyphenyl-1,3,5-triazine compound of general formula (I-m)

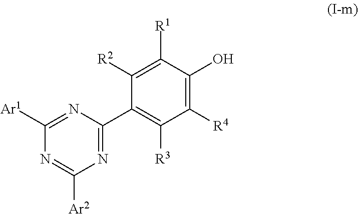

wherein
Ar¹ and Ar² are identical or different and independently selected from substituted or unsubstituted C₆-C₁₈ aryl, or a moiety of the formula (A),

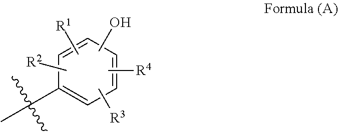

wherein
R¹ to R⁴ are independently selected from hydrogen, —OH, —CN, —COOH, substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, substituted or unsubstituted C₇-C₂₄ arylalkyl, OR⁵, COOR⁵, NR⁵R⁶, or C(═O)NR⁵R⁶, wherein R⁵ is a linear or branched, substituted or unsubstituted C₁-C₁₈ alkyl, and R⁶ is a linear or branched, substituted or unsubstituted C₁-C₁₈ alkyl.

The process comprises the steps of
a) preparing a deep eutectic mixture (DEM) (IV-m) by mixing at least one quaternary ammonium salt of general formula (II) and at least one phenol of general formula (Ill-m);

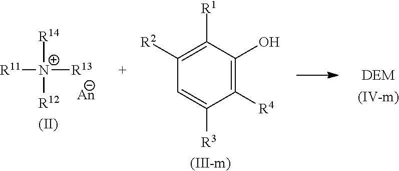

wherein R¹¹ to R¹⁴ are independently selected from linear or branched, substituted or unsubstituted C₁-C₁₈ alkyl, or substituted or unsubstituted C₇-C₂₄ arylalkyl, or
R¹¹ and R¹² together with the nitrogen atom to which they are bonded form a substituted or unsubstituted, saturated or unsaturated 5- to 12-membered cycloalkyl ring that optionally contains 1, 2 or 3 heteroatom(s) selected from O, N, or S as ring member(s); and An⁻ is selected from chloride, bromide, iodide, fluoride, sulfate, sulfonate, alkylsulfate, or carboxylate, and R¹ to R⁴ as defined above; and
b) reacting at least one substituted 2-chloro-1,3,5-triazine compound of general formula (V) and the deep eutectic mixture obtained according to step (a) to obtain the hydroxyphenyl-1,3,5-triazine compound of general formula (I-m);

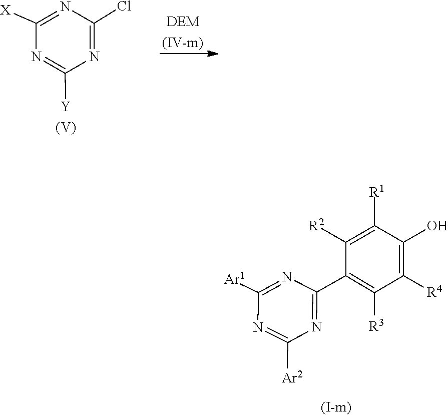

wherein
X is chloro or Ar¹, and Y is chloro or Ar².

In a more preferred embodiment, the presently claimed invention is directed to a process for preparing a hydroxyphenyl-1,3,5-triazine compound of general formula (I-n)

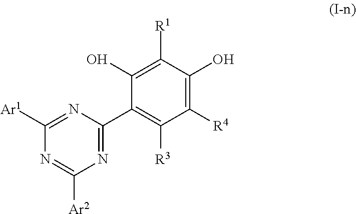

wherein
Ar¹ and Ar² are identical or different and independently selected from substituted or unsubstituted C₆-C₁₈ aryl, or a moiety of the formula (A),

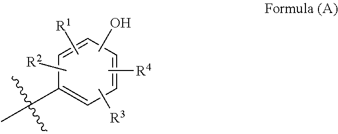

wherein
R¹ to R⁴ are independently selected from hydrogen, —OH, —CN, —COOH, substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, substituted or unsubstituted C₇-C₂₄ arylalkyl, OR⁵, COOR⁵, NR⁵R⁶, or C(═O)NR⁵R⁶, wherein R⁵ is a linear or branched, substituted or unsubstituted C₁-C₁₈ alkyl, and R⁶ is a linear or branched, substituted or unsubstituted C₁-C₁₈ alkyl.

The process comprises the steps of
a) preparing a deep eutectic mixture (DEM) (IV-n) by mixing at least one quaternary ammonium salt of general formula (II) and at least one phenol of general formula (Ill-n);

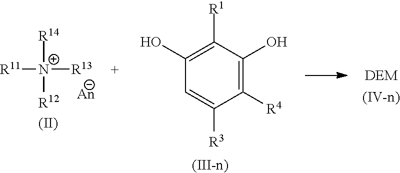

wherein R¹¹ to R¹⁴ are independently selected from linear or branched, substituted or unsubstituted C₁-C₁₈ alkyl, or substituted or unsubstituted C₇-C₂₄ arylalkyl, or
R¹¹ and R¹² together with the nitrogen atom to which they are bonded form a substituted or unsubstituted, saturated or unsaturated 5- to 12-membered cycloalkyl ring that optionally contains 1, 2 or 3 heteroatom(s) selected from O, N, or S as ring member(s); and An⁻ is selected from chloride, bromide, iodide, fluoride, sulfate, sulfonate, alkylsulfate, or carboxylate, and R¹ to R⁴ as defined above; and
b) reacting at least one substituted 2-chloro-1,3,5-triazine compound of general formula (V) and the deep eutectic mixture obtained according to step (a) to obtain the hydroxyphenyl-1,3,5-triazine compound of general formula (I-n);

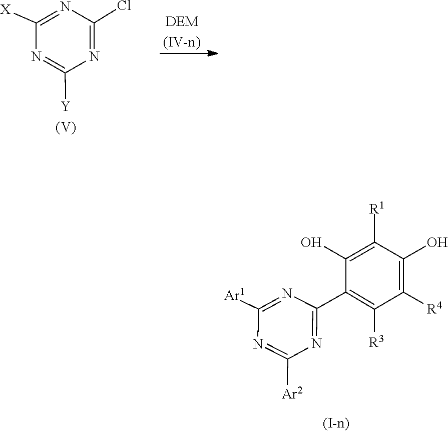

wherein
X is chloro or Ar¹, and Y is chloro or Ar².

In a preferred embodiment, the molar ratio of the at least one quaternary ammonium salt (II) and the at least one phenol (III) is in the range of 1:1 to 1:15.

In a more preferred embodiment, the molar ratio of the at least one quaternary ammonium salt (II) and the at least one phenol (III) is in the range of 1:2 to 1:10; even preferably in the range of 1:3 to 1:8; and most preferably in the range of 1:3 to 1:5.

In a particularly preferred embodiment, the molar ratio of the at least one quaternary ammonium salt (II) and the at least one phenol (III) is 1:4.

In a preferred embodiment, in step (a) the mixture comprising the at least one quaternary ammonium salt (II) and the at least one phenol (III) is heated at a temperature in the range of 20 to 150° C. for a time in the range of 5 min to 12 hours.

In a more preferred embodiment, in step (a) the mixture comprising the at least one quaternary ammonium salt (II) and the at least one phenol (III) is mixed at a temperature in the range of 40 to 150° C.; even more preferably at a temperature in the range of 60 to 125° C.; and most preferably at a temperature in the range of 80 to 120° C.

In a particularly preferred embodiment, in step (a) the mixture comprising the at least one quaternary ammonium salt (II) and the at least one phenol (III) is mixed at a temperature in the range of 90 to 110° C.

In a preferred embodiment, in step (a) the mixture comprising the at least one quaternary ammonium salt (II) and the at least one phenol (III) is mixed for a time in the range of 5 min to 300 min; more preferably 10 min to 240 min; and most preferably 15 min to 120 min.

In a preferred embodiment, step (a) is carried out without the use of a solvent.

The at least one quaternary ammonium salt and the at least one phenol form a clear DEM (IV). Solvent is not required for this step. The step of formation of DEM proceeds without the use of a solvent even if, either one or both of the (II) and (III) is/are solid.

Step (a) can also be carried out in a solvent selected from toluene, o-dichlorobenzene, chlorobenzene, acetonitrile, methyl ethyl ketone, methyl THF, or methyl t-butyl ether. The catalytic activity of the DEM is not significantly affected by the presence of solvent.

In a preferred embodiment, R¹¹ to R¹⁴ are independently selected from linear or branched, substituted or unsubstituted C₁-C₂₄ alkyl; more preferably C₁-C₁₈ alkyl; even more preferably C₁-C₁₂ alkyl; and most preferably C₁-C₈ alkyl.

In a preferred embodiment, R¹¹ to R¹³ are independently selected from linear or branched, substituted or unsubstituted C₁-C₂₄ alkyl; more preferably C₁-C₁₈ alkyl; even more preferably C₁-C₁₂ alkyl; and most preferably C₁-C₈ alkyl; and
R¹⁴ is selected from —OH substituted, linear or branched C₁-C₂₄ alkylene; more preferably C₁-C₁₂ alkylene; even more preferably C₁-C₆ alkylene; and most preferably C₁-C₄ alkylene.

In a preferred embodiment, R¹¹ and R¹² are independently selected from linear or branched, substituted or unsubstituted C₁-C₂₄ alkyl; more preferably C₁-C₁₈ alkyl; even more preferably C₁-C₁₂ alkyl; and most preferably C₁-C₈ alkyl;
R¹³ is selected from substituted or unsubstituted C₇-C₂₄ arylalkyl; more preferably C₇-C₁₂ alkyl; and most preferably benzyl; and
R¹⁴ is selected from —OH substituted, linear or branched C₁-C₂₄ alkylene; more preferably C₁-C₁₂ alkylene; even more preferably C₁-C₆ alkylene; and most preferably C₁-C₄ alkylene.

In a more preferred embodiment, R¹¹ to R¹³ are independently selected from linear or branched, substituted or unsubstituted C₁-C₈ alkyl, or benzyl, and
R¹⁴ is selected from —OH substituted, linear or branched C₁-C₈ alkylene.

In a preferred embodiment, R¹¹ and R¹² together with the nitrogen atom to which they are bonded form substituted or unsubstituted, saturated or unsaturated 5- to 6-membered cycloalkyl ring that optionally contains one heteroatom selected from O, N, or S as ring member(s),
R¹³ is linear or branched, substituted or unsubstituted C₁-C₂₄ alkyl, and
R¹⁴ is selected from linear or branched, substituted or unsubstituted C₁-C₆ alkyl.

In a preferred embodiment, the An⁻ is selected from chloride, bromide, iodide, fluoride, sulfate, sulfonate, methylsulfate, ethylsulfate, salicylate, or acetate.

In a preferred embodiment, the at least one quaternary ammonium salt of formula (II) is selected from choline chloride, choline fluoride, choline bromide, choline iodide, choline acetate, choline salicylate, tetra-n-ethylammonium chloride, tetramethylammonium chloride, tetra-n-butylammonium chloride, N,N-diethyl ethanol ammonium chloride, N-benzyl-2-hydroxy-N,N-dimethylethanaminium chloride, N-ethyl-2-hydroxy-N,N-dimethylethanaminium chloride, 2-(chlorocarbonyloxy)-N,N,N-trimethylethenaminium chloride, N-methyl-N,N,N-trioctyl ammonium chloride, tris(2-hydroxyethyl)methylammonium methyl sulfate, or tributylmethylammonium methyl sulfate or cetethyl morpholinium ethane sulfate.

In a more preferred embodiment, the at least one quaternary ammonium salt of formula (II) is choline chloride.

In a preferred embodiment, the at least one phenol of formula (III) is selected from substituted or unsubstituted phenol, resorcinol, catechol, quinol, cresol, methoxyphenol, salicylic acid, α-naphthol, or β-naphthol.

In a more preferred embodiment, the at least one phenol of formula (III) is resorcinol.

In a preferred embodiment, the deep eutectic mixture (IV) comprises choline chloride and resorcinol.

In a preferred embodiment, the molar ratio of choline chloride and resorcinol is in the range of 1:1 to 1:15.

In a more preferred embodiment, the molar ratio of choline chloride and resorcinol is in the range of 1:3 to 1:8; even more preferably 1:3 to 1:6; and most preferably 1:4.

In a more preferred embodiment, the at least one quaternary ammonium salt of formula (II) is cetethyl morpholinium ethane sulfate.

In a preferred embodiment, the substituted 2-chloro-1,3,5-triazine compound of formula (V) is selected from cyanuric chloride, 6-aryl-2,4-dichloro-1,3,5-triazine, or 4,6-diaryl-2-chloro-1,3,5-triazine.

In a more preferred embodiment, the at least one substituted 2-chloro-1,3,5-triazine compound of formula (V) is cyanuric chloride.

In a preferred embodiment, the at least one substituted 2-chloro-1,3,5-triazine compound of formula (V) is 6-aryl-2,4-dichloro-1,3,5-triazine.

In a more preferred embodiment, the at least one substituted 6-aryl-2,4-dichloro-1,3,5-triazine compound of formula (V) is 2,4-dichloro-6-(4-methoxy)phenyl-1,3,5-triazine.

In a preferred embodiment, the molar ratio of the at least one substituted 2-chloro-1,3,5-triazine compound of formula (V) and the at least one phenol (III) is in the range of 1:2 to 1:20.

In a more preferred embodiment, the molar ratio of the at least one substituted 2-chloro-1,3,5-triazine compound of formula (V) and the at least one phenol (III) is in the range of 1:2 to 1:15; even more preferably in the range of 1:2 to 1:10; and most preferably 1:2 and 1:6.

In a preferred embodiment, the weight ratio of the at least one substituted 2-chloro-1,3,5-triazine compound of formula (V) and the DEM (IV) is in the range of 1:2 to 1:10.

In a more preferred embodiment, the weight ratio of the at least one substituted 2-chloro-1,3,5-triazine compound of formula (V) and the DEM (IV) is in the range of 1:2 to 1:8.

In a particularly preferred embodiment, the weight ratio of the at least one substituted 2-chloro-1,3,5-triazine compound of formula (V) and the DEM (IV) is 1:4.

In a preferred embodiment, step (b) involves a step of addition of the at least one substituted 2-chloro-1,3,5-triazine compound of general formula (V) to DES (IV) at a temperature in the range of 30 to 150° C. over a period in the range of 5 min to 12 hours.

In a more preferred embodiment, the addition of the at least one substituted 2-chloro-1,3,5-triazine compound of general formula (V) to DES (IV) is carried out at a temperature in the range of 50 to 125° C.; and most preferably 80 to 115° C.

In a more preferred embodiment, the addition of the at least one substituted 2-chloro-1,3,5-triazine compound of general formula (V) to DES (IV) is carried out over a period in the range of 5 min to 600 min; even more preferably 15 min to 300 min; and most preferably 15 min to 120 min.

In a preferred embodiment, in step (b), the reaction mixture obtained upon complete addition of the at least one substituted 2-chloro-1,3,5-triazine compound of formula (V) to DES (IV) is heated at a temperature in the range of 30 to 150° C. for 5 min to 300 min.

In a more preferred embodiment, in step (b), the reaction mixture is heated at a temperature in the range of 50 to 130° C.; and most preferably 80 to 120° C.

In a more preferred embodiment, in step (b), the reaction mixture is heated for 10 min to 180 min; and most preferably for 10 min to 120 min.

In a preferred embodiment, step (b) is carried out without the use of a solvent.

The starting material 2-chloro-1,3,5-triazine compound of general formula (V) and product hydroxyphenyl-1,3,5-triazine compound of formula (I) have a low solubility. The DEM (IV) acts as a solvent for both the starting material and the product. Therefore, solvent is not required for step (b).

Step (b) can also be carried out in a solvent selected from toluene, o-dichlorobenzene, chlorobenzene, acetonitrile, methyl ethyl ketone, methyl THF, or methyl t-butyl ether.

In a preferred embodiment, the hydroxyphenyl-1,3,5-triazine compound of formula (I) is

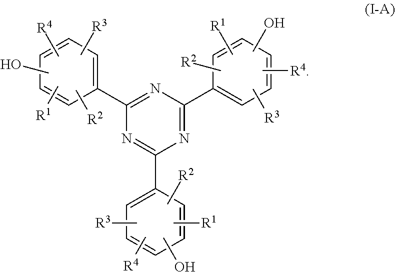

In a preferred embodiment, the hydroxyphenyl-1,3,5-triazine compound of formula (I) is 2,4,6-tris(2,4-dihydroxyphenyl)-triazine (VI).

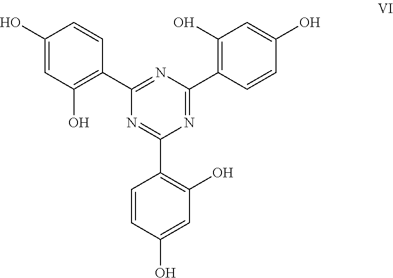 [SMILES: Oc1ccc(-c2nc(-c3ccc(O)cc3O)nc(-c3ccc(O)cc3O)n2)c(O)c1]

In a preferred embodiment, the hydroxyphenyl-1,3,5-triazine compound of formula (I) is

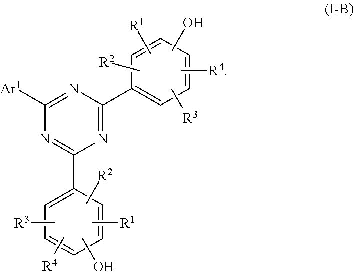

In a preferred embodiment, the hydroxyphenyl-1,3,5-triazine compound of formula (I) is 2-(4-methoxyphenyl)-4,6-bis-(2,4-dihydroxyphenyl)-1,3,5-triazine (VII)

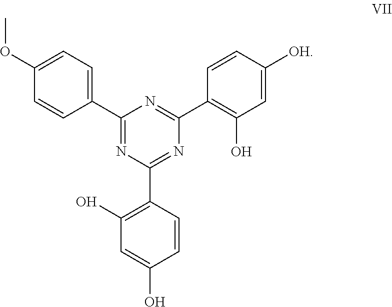 [SMILES: COc1ccc(-c2nc(-c3ccc(O)cc3O)nc(-c3ccc(O)cc3O)n2)cc1]

In a preferred embodiment, the process of the present invention further comprises a step of quenching the reaction by adding water to the reaction mixture.

In a preferred embodiment, the process comprises separating the aqueous phase comprising the at least one quaternary ammonium salt of formula (II) and the at least one phenol of formula (III).

In a preferred embodiment, the process comprises recovery of the at least one phenol of formula (III) from the aqueous phase.

In a more preferred embodiment, at least one phenol of formula (III) is recovered from the aqueous phase by using at least one technique selected from the evaporation of water, or extraction of the aqueous phase with an organic solvent.

In a most preferred embodiment, the at least one phenol of formula (III) is recovered from the aqueous phase by extraction of the aqueous phase with methyl-TH F.

It is found that the recovered phenol can be reused for preparation of DEM (IV) without loss of activity of DEM (IV).

In a preferred embodiment, the at least one phenol of formula (III) comprises recovered at least one phenol of formula (III).

In a preferred embodiment, the yield of the hydroxyphenyl-1,3,5-triazine compound of formula (I) is in the range of 40 to 80 wt %; more preferably 45 to 70 wt %; and most preferably 50 to 60 wt %.

In a preferred embodiment, the purity of the hydroxyphenyl-1,3,5-triazine compound of formula (I) is in the range of 80 to 99.9%.

In a preferred embodiment, the selectivity of the hydroxyphenyl-1,3,5-triazine compound of formula (I) is in the range of 60 to 90%.

Another aspect of the presently claimed invention is directed to use of the hydroxyphenyl-1,3,5-triazine compound of formula (I) obtained by the process of the present invention to prepare 2,4-bis(2-hydroxy-4-butoxyphenyl)-6-(2,4-dibutoxyphenyl)-1,3,5-triazine, 2,4,6-tris(2-hydroxy-4-isooctyloxycarbonylethylideneoxyphenyl)triazine, and bis-ethylhexyloxyphenol methoxyphenyl triazine.

The process of the presently claimed invention has a high space time yield and volume yield because of one or more of the following reasons:
(i) The process is carried out without the use of a solvent and the reaction mixture comprises reactants and quaternary ammonium salt. As a result, the space requirement for the reaction is low.
(ii) The work up is easy and involves a simple step of addition of water to the reaction mixture followed by stirring.
(iii) The process is fast with low reaction time in both steps.

The presently claimed invention offers one or more of the following advantages:
1) DEM acts as a non-metal catalyst for the Friedel-Craft alkylation of 2-chloro-1,3,5-triazine compound of general formula (V) and at least one phenol of general formula (III).
2) DEM acts as a green solvent for the reaction. Therefore, the Friedel-Craft alkylation of (V) and (III) can be carried out without the use of a solvent.
3) The process of the present invention involves easy quenching as well as work-up procedure.
4) The process of the present invention has a high space time yield and volume yield.
5) The process of the present invention involves recovery of phenol, which can be reused.
6) The process of the present invention is economical.

In the following, there are provided a list of embodiments to further illustrate the present disclosure without intending to limit the disclosure to specific embodiments listed below.
1. A process for preparing a hydroxyphenyl-1,3,5-triazine compound of general formula (I)

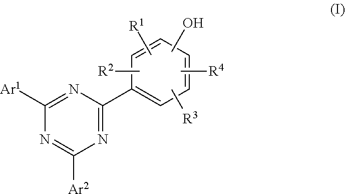

wherein
Ar¹ and Ar² are identical or different and independently selected from substituted or unsubstituted C₆-C₁₈ aryl, or a moiety of the formula (A),

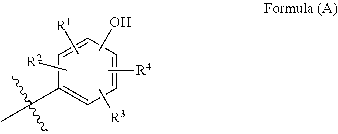

wherein
R¹ to R⁴ are independently selected from hydrogen, —OH, —CN, —COOH, substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, substituted or unsubstituted C₇-C₂₄ arylalkyl, OR⁵, COOR⁵, NR⁵R⁶, or C(═O)NR⁵R⁶, wherein R⁵ is a linear or branched, substituted or unsubstituted C₁-C₁₈ alkyl, and R⁶ is a linear or branched, substituted or unsubstituted C₁-C₁₈ alkyl,
the process comprising the steps of
a) preparing a deep eutectic mixture (DEM) (IV) by mixing at least one quaternary ammonium salt of general formula (II) and at least one phenol of general formula (III);

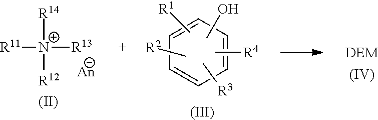

wherein R¹¹ to R¹⁴ are independently selected from linear or branched, substituted or unsubstituted C₁-C₁₈ alkyl, or substituted or unsubstituted C₇-C₂₄ arylalkyl, or
R¹¹ and R¹² together with the nitrogen atom to which they are bonded form a substituted or unsubstituted, saturated or unsaturated 5- to 12-membered cycloalkyl ring that optionally contains 1, 2 or 3 heteroatom(s) selected from O, N, or S as ring member(s); and An⁻ is selected from chloride, bromide, iodide, fluoride, sulfate, sulfonate, alkylsulfate, or carboxylate, and R¹ to R⁴ as defined above; and
b) reacting at least one substituted 2-chloro-1,3,5-triazine compound of general formula (V) and the deep eutectic mixture obtained according to step (a) to obtain the hydroxyphenyl-1,3,5-triazine compound of general formula (I);

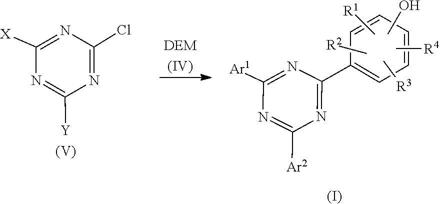

wherein
X is chloro or Ar¹, and Y is chloro or Ar².
2. The process according to embodiment 1, wherein the molar ratio of the at least one quaternary ammonium salt (II) and the at least one phenol (III) is in the range of 1:1 to 1:15.
3. The process according to embodiment 1 or 2, wherein in step (a) the mixture comprising the at least one quaternary ammonium salt (II) and the at least one phenol (III) is mixed at a temperature in the range of 20 to 150° C. for a time in the range of 5 min to 12 hours.
4. The process according to any of embodiments 1 to 3, wherein step (a) is carried out without the use of a solvent.
5. The process according to any of embodiments 1 to 3, wherein step (a) is carried out in a solvent selected from toluene, o-dichlorobenzene, chlorobenzene, acetonitrile, methyl ethyl ketone, methyl TH F, or methyl t-butyl ether.
6. The process according to any of embodiments 1 to 5, wherein R¹¹ to R¹⁴ are independently selected from linear or branched, substituted or unsubstituted C₁-C₂₄ alkyl, or substituted or unsubstituted C₇-C₂₄ arylalkyl.
7. The process according to any of embodiments 1 to 6, wherein
   R¹¹ to R¹³ are independently selected from linear or branched, substituted or unsubstituted C₁-C₈ alkyl, or substituted or unsubstituted C₇-C₁₀ arylalkyl, and
   R¹⁴ is selected from —OH substituted, linear or branched C₁-C₈ alkylene.
8. The process according to any of embodiments 1 to 6, wherein
   R¹¹ and R¹² together with the nitrogen atom to which they are bonded form substituted or unsubstituted, saturated or unsaturated 5- to 6-membered cycloalkyl ring that optionally contains one heteroatom selected from O, N, or S as ring member,
   R¹³ is linear or branched, substituted or unsubstituted C₁-C₂₄ alkyl, and
   R¹⁴ is selected from linear or branched, substituted or unsubstituted C₁—C alkyl.
9. The process according to any of claims 1 to 6, wherein the An⁻ is selected from chloride, bromide, iodide, fluoride, sulfate, sulfonate, methylsulfate, ethylsulfate, salicylate, or acetate.
10. The process according to any of embodiments 1 to 8, wherein the at least one quaternary ammonium salt of formula (II) is selected from choline chloride, choline fluoride, choline bromide, choline iodide, choline acetate, choline salicylate, tetra-n-ethyl ammonium chloride, tetramethylammonium chloride, tetra-n-butylammonium chloride, N,N-diethyl ethanol ammonium chloride, N-benzyl-2-hydroxy-N,N-dimethylethanaminium chloride, N-ethyl-2-hydroxy-N,N-dimethylethanaminium chloride, 2-(chloro carbonyloxy)-N,N,N-trimethylethenaminium chloride, N-methyl-N,N,N-trioctyl ammonium chloride, tris(2-hydroxyethyl)methylammonium methyl sulfate, or tributylmethylammonium methyl sulfate or cetethyl morpholinium ethane sulfate.
11. The process according to any of embodiments 1 to 9, wherein the at least one quaternary ammonium salt of formula (II) is choline chloride.
12. The process according to any of embodiments 1 to 10, wherein the at least one phenol of formula (III) is selected from substituted or unsubstituted phenol, resorcinol, catechol, quinol, cresol, methoxyphenol, salicylic acid, α-naphthol, or β-naphthol.
13. The process according to any of embodiments 1 to 11, wherein the deep eutectic mixture (IV) comprises choline chloride and resorcinol.
14. The process according to embodiment 12, wherein the molar ratio of choline chloride and resorcinol is in the range of 1:1 to 1:15.
15. The process according to embodiment 11 or 12, wherein the molar ratio of choline chloride and resorcinol is in the range of 1:3 to 1:6.
16. The process according to any of embodiment 11 to 13, wherein the molar ratio of choline chloride and resorcinol is 1:4.
17. The process according to any of embodiments 1 to 16, wherein the substituted 2-chloro-1,3,5-triazine compound of formula (V) is selected from cyanuric chloride, 6-aryl-2,4-dichloro-1,3,5-triazine, or 4,6-diaryl-2-chloro-1,3,5-triazine.
18. The process according to any of embodiments 1 to 17, wherein the at least one substituted 2-chloro-1,3,5-triazine compound of formula (V) is cyanuric chloride.
19. The process according to any of embodiments 1 to 17, wherein the at least one substituted 2-chloro-1,3,5-triazine compound of formula (V) is 6-aryl-2,4-dichloro-1,3,5-triazine.
20. The process according to embodiment 19, wherein the at least one substituted 2-chloro-1,3,5-triazine compound of formula (V) is 2,4-dichloro-6-(4-methoxy)phenyl-1,3,5-triazine.
21. The process according to any of embodiments 1 to 20, wherein the molar ratio of the at least one substituted 2-chloro-1,3,5-triazine compound of formula (V) and the at least one phenol (III) is in the range of 1:2 to 1:20.
22. The process according to embodiment 21, wherein the molar ratio of the at least one substituted 2-chloro-1,3,5-triazine compound of formula (V) and the at least one phenol (III) is in the range of 1:2 to 1:15.
23. The process according to any of embodiments 1 to 22, wherein step (b) involves a step of addition of the at least one substituted 2-chloro-1,3,5-triazine compound of general formula (V) to DES (IV) at a temperature in the range of 30 to 150° C. over a period in the range of 5 min to 12 hours.
24. The process according to any of embodiments 1 to 23, wherein in step (b), the reaction mixture obtained upon complete addition of the at least one substituted 2-chloro-1,3,5-triazine compound of formula (V) to DES (IV) is heated at a temperature in the range of 30 to 150° C. for 5 min to 600 min.
25. The process according to any of embodiments 1 to 24, wherein step (b) is carried out without the use of a solvent.
26. The process according to any of embodiments 1 to 24, wherein step (b) is carried out in a solvent selected from toluene, o-dichlorobenzene, chlorobenzene, acetonitrile, methyl ethyl ketone, methyl THF, or methyl t-butyl ether.
27. The process according to any of embodiments 1 to 26, wherein the hydroxyphenyl-1,3,5-triazine compound of formula (I) is

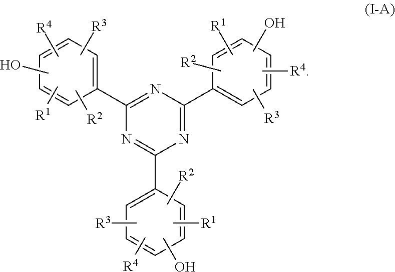

28. The process according to embodiment 27, wherein the hydroxyphenyl-1,3,5-triazine compound of formula (I) is 2,4,6-tris(2,4-dihydroxyphenyl)-triazine (VI)

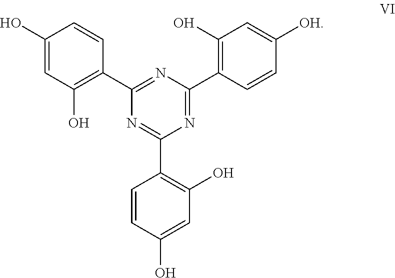 [SMILES: Oc1ccc(-c2nc(-c3ccc(O)cc3O)nc(-c3ccc(O)cc3O)n2)c(O)c1]

29. The process according to any of embodiments 1 to 26, wherein the hydroxyphenyl-1,3,5-triazine compound of formula (I) is

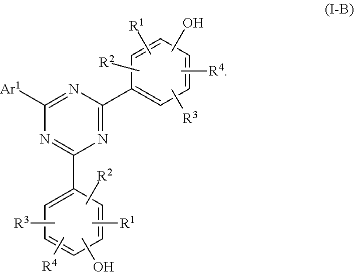

30. The process according to embodiment 29, wherein the hydroxyphenyl-1,3,5-triazine compound of formula (I) is 2-(4-methoxyphenyl)-4,6-bis-(2,4-dihydroxyphenyl)-1,3,5-triazine (VII).

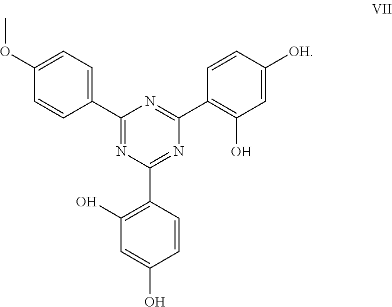 [SMILES: COc1ccc(-c2nc(-c3ccc(O)cc3O)nc(-c3ccc(O)cc3O)n2)cc1]

31. The process according to any of embodiments 1 to 30 further comprises a step of quenching the reaction by adding water to the reaction mixture.
32. The process according to embodiment 31 comprises separating the aqueous phase comprising the at least one quaternary ammonium salt of formula (II) and the at least one phenol of formula (III).
33. The process according to embodiment 32 comprises recovery of the at least one phenol of formula (III) from the aqueous phase.
34. The process according to any of embodiments 1 to 33, wherein the at least one phenol of formula (III) comprises recovered at least one phenol of formula (III).
35. The process according to any of embodiments 1 to 34, wherein the yield of the hydroxyphenyl-1,3,5-triazine compound of formula (I) is in the range of 40 to 80 wt. %.
36. The process according to any of embodiments 1 to 35, wherein the purity of the hydroxyphenyl-1,3,5-triazine compound of formula (I) is in the range of 80 to 99.9%.
37. The process according to any of embodiments 1 to 36, wherein the selectivity of the hydroxyphenyl-1,3,5-triazine compound of formula (I) is in the range of 60 to 90%.
38. Use of the hydroxyphenyl-1,3,5-triazine compound of formula (I) obtained by the process according to any one of embodiments 1 to 37 to prepare 2,4-bis(2-hydroxy-4-butoxyphenyl)-6-(2,4-dibutoxyphenyl)-1,3,5-triazine, 2,4,6-tris(2-hydroxy-4-isooctyloxycarbonylethylideneoxyphenyl)triazine, and bis-ethylhexyloxyphenol methoxyphenyl triazine.

While the presently claimed invention has been described in terms of its specific embodiments, certain modifications and equivalents will be apparent to those skilled in the art and are intended to be included within the scope of the presently claimed invention.

### EXAMPLES

The presently claimed invention is illustrated in detail by non-restrictive working examples which follow. More particularly, the test methods specified hereinafter are part of the general disclosure of the application and are not restricted to the specific working examples.

### Materials

The following materials are used in the Examples:

Choline chloride is available from TCI Chemicals.

Resorcinol is available from Sigma Aldrich Chemical Pvt. Ltd.

Cyanuric chloride is available from Avra Synthesis Pvt. Ltd.

Dimethyl formamide (DMF) is available from Spectrochem Pvt. Ltd.

### Example 1: Preparation of 2,4,6-tris(2,4-dihydroxyphenyl)-triazine (VI)

(Choline chloride:resorcinol mole ratio—1:4, and Cyanuric chloride:DEM weight ratio—1:6)

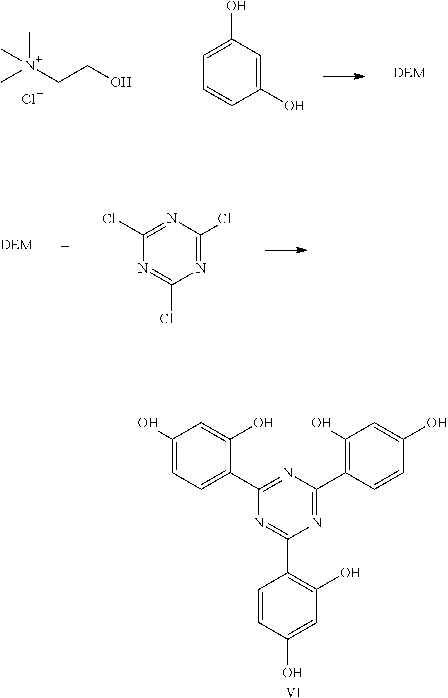

To a 250 mL jacketed glass reactor maintained under nitrogen atmosphere were charged choline chloride 7.2 g (51.6 mmol) and resorcinol 22.8 g (207.1 mmol), and the mixture was heated at 95° C. for 30 min to get a clear solution of DEM.

Cyanuric chloride 5.0 g (27.1 mol) was slowly added to the reactor over a period of 120 min, while maintaining temperature of the reaction mixture at 110° C. After complete addition, reaction mixture was stirred at 110° C. for 240 min, followed by cooling to 50° C.

Water (200 g) was slowly added to the reaction mass to obtain a suspension containing yellow precipitate. The suspension was stirred for 30 min at 23° C. The suspension was filtered to obtain a filter cake and a filtrate comprising the aqueous phase. The filter cake was washed with water (2×100 g) to obtain VI. (Weight-8.4 g (76.36%). HPLC Purity-65.80%).

### Examples 2-5: Effect of Weight Ratio of Choline Chloride and Resorcinol

2,4,6-Tris(2,4-dihydroxyphenyl)-triazine (VI) was prepared at varying molar ratio of choline chloride and resorcinol, while maintaining the weight ratio of 2-chloro-1,3,5-triazine compound and DEM 1:6. The experiments were carried out using the experimental procedure of Example 1.

The results are summarized in Table 1.

TABLE 1

| | Choline | | Molar | | | Weight ratio | | |
|---|---|---|---|---|---|---|---|---|
| | chloride | Resorcinol | ratio | | Cyanuric | cyanuric | % | Purity |
| Ex. | (ChCh) | (Res) | ChCh:Res | DEM | chloride | chloride:DEM | Yield | (%) |
| 1 | 7.2 g | 22.8 g | 1:4 | 30 g | 5.0 g | 1:6 | 76.36 | 65.80 |
| 2 | 16.8 g | 13.2 g | 1:1 | 30 g | 5.0 g | 1:6 | 0.69* | — |
| 3 | 11.6 g | 18.4 g | 1:2 | 30 g | 5.0 g | 1:6 | 9.09 | 65.00 |
| 4 | 9.0 g | 21.0 g | 1:3 | 30 g | 5.0 g | 1:6 | 62.73 | 55.75 |
| 5 | 24.0 g | 96.0 g | 1:5 | 120 g | 20.0 g | 1:6 | 56.86 | 64.03 |

*HPLC conversion

The use of DEM comprising choline chloride and resorcinol in a molar ratio of 1:1 and 1:2 provided product with a low yield. The use DEM comprising choline chloride and resorcinol in a molar ratio of 1:3 and higher provided the product with high yield.

### Example 6: Preparation of 2,4,6-tris(2,4-dihydroxyphenyl)-triazine (VI) including purification

(Choline chloride:resorcinol mole ratio—1:4, and Cyanuric chloride:DEM weight ratio—1:4)

To 500 mL jacketed glass reactor maintained under nitrogen atmosphere were charged choline chloride 19.0 g (143.2 mmol) and resorcinol 61.0 g (544.9 mmol), and the mixture was heated at 95° C. for 30 min to get a clear solution of DEM.

Cyanuric chloride 20.0 g (108.4 mol) was slowly added to the reactor over a period of 120 min, while maintaining temperature of the reaction mixture at 110° C. After complete addition, the reaction mixture was stirred at 110° C. for 240 min, followed by cooling to 50° C.

Water (200 g) was slowly added to the reaction mass to obtain a suspension containing yellow precipitate. The suspension was stirred for 40 min at 23° C. The suspension was filtered to obtain a filter cake and a filtrate comprising the aqueous phase. The filter cake was washed with water (2×100 g) to obtain wet cake containing VI.

The wet cake was transferred to 1 L vessel followed by addition of 200 ml DMF. The content was stirred at 100° C. for 60 min and gradually cooled to 10° C., followed by stirring for 40 min to allow complete precipitation. The suspension was filtered to obtain the yellow solid which was washed with cold methanol (100 ml). The product was dried under reduced pressure at 75° C. to obtain VI (Weight-24 g (54.5%).

HPLC Purity: 97%

Selectivity: 70.4% (by HPLC)

¹H-NMR (500 MHz, d₆-DMSO): δ 12.89 (s, 3H), 10.47 (s, 3H), 8.10 (d, 3H), 6.53 (dd, 6H), 6.41 (d, 3H)

¹³C-NMR (500 MHz, d₆-DMSO): δ 168.31, 164.52, 163.82, 131.53, 109.46, 109.24, 103.74.

### Example 7: Preparation of 2,4,6-tris(2,4-dihydroxyphenyl)-triazine (VI) including purification

(Choline chloride:resorcinol mole ratio—1:4, and Cyanuric chloride:DEM weight ratio—1:6)

The reaction was carried out using the procedure of example 6 at the cyanuric acid to DEM weight ratio of 1:6. The amounts of materials were as follows: choline chloride 58.0 g (415.0 mmol), resorcinol 182.0 g (165.3 mmol) and cyanuric chloride 40.0 g (216.0 mmol).

The amount of compound VI obtained after recrystallization was 45.0 g (51.1%) and HPLC purity 98.5%.

### Examples 8-9: Effect of molar ratio of 2-chloro-1,3,5-triazine compound and DEM

2,4,6-Tris(2,4-dihydroxyphenyl)-triazine (VI) was prepared using different weight ratios of 2-chloro-1,3,5-triazine compound and DEM while keeping the mole ratio of choline chloride to resorcinol at 1:4. The experiments were carried out using the experimental procedure of Example 1.

The step of recrystallization was not carried out for examples 8 and 9. Thus, the filter cake obtained after water-wash was dried to obtain (VI).

The results are summarized in Table 2.

TABLE 2

| | | | | | Weight ratio | | |
|---|---|---|---|---|---|---|---|
| | Choline | | Weight | Cyanuric | Cyanuric | % | Purity |
| Example | chloride | Resorcinol | of DEM | chloride | chloride:DEM | Yield | (%) |
| 1 | 7.2 g | 22.8 g | 30.0 g | 5.0 g | 1:6 | 76.36 | 65.80 |
| 8 | 6.0 g | 18.0 g | 24.0 g | 5.0 g | 1:5 | 68.18 | 59.14 |
| 9 | 1.0 g | 3.15 g | 4.15 g | 0.5 g | 1:8 | 74.1 | 57.13 |

*conversion by HPLC

### Example 8: Effect of Reaction Time

2,4,6-Tris(2,4-dihydroxyphenyl)-triazine (VI) was prepared using the experimental procedure of Example 1 except that the reaction was carried out for 22 hours instead of 5 hours.

The yield of crude product was 66.36% with a purity of 72.16%.

### Example 9: Recovery of Resorcinol from Example 1

The filtrate comprising aqueous phase obtained in Example 6 was subjected to recovery of resorcinol.

The filtrate was extracted with methyl-THF (2×200 ml). The combined organic layer was dried using sodium sulphate and concentrated under reduced pressure to obtain product containing recovered resorcinol, Weight-35 g (HPLC purity-Resorcinol −76.5%) (Actual yield on Assay basis-26.4 g).

The aqueous layer obtained after extraction with methyl-THF was concentrated under reduced pressure to obtain product containing recovered resorcinol, Weight-17 g (moisture-8.8%).

### Experiment 10: Preparation of Compound (VI) Using Recovered Resorcinol

Compound (VI) was prepared using a procedure similar to that of experiment 1.

Recovered resorcinol (organic extraction) 26.0 g (254.3 mmol, 5.0 eq) from experiment 9 was used for this experiment. The amount of choline chloride was 9.0 g (64.46 mmol, 1.3 eq) and the amount of cyanuric chloride was 6.0 g (32.5 mmol, 1.0 eq).

The amount of (VI) obtained was 9.0 g, yield—68.2% and HPLC Purity—53.2%.

1.-24. (canceled)

25. A process for preparing a hydroxyphenyl-1,3,5-triazine compound of general formula (I), 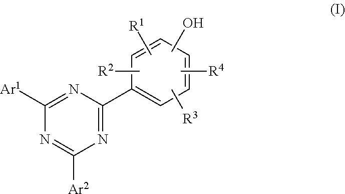
wherein
Ar¹ and Ar² are identical or different and independently selected from substituted or unsubstituted C₆-C₁₈ aryl, or a moiety of the formula (A), 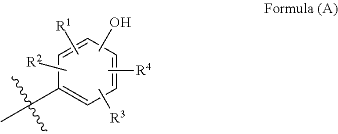
wherein
R¹ to R⁴ are independently selected from hydrogen —OH, —CN, —COOH, substituted or unsubstituted, linear or branched C₁-C₂₄ alkyl, substituted or unsubstituted, linear or branched C₂-C₂₄ alkenyl, substituted or unsubstituted C₅-C₂₄ cycloalkyl, substituted or unsubstituted C₅-C₂₄ cycloalkenyl, substituted or unsubstituted C₆-C₂₄ aryl, substituted or unsubstituted C₇-C₂₄ arylalkyl, OR⁵, COOR⁵, NR⁵R⁶, or C(═O)NR⁵R⁶, wherein R⁵ is a linear or branched, substituted or unsubstituted C₁-C₁₈ alkyl, and R⁶ is a linear or branched, substituted or unsubstituted C₁-C₁₈ alkyl,
the process comprising the steps of
a) preparing a deep eutectic mixture (DEM) (IV) by mixing at least one quaternary ammonium salt of general formula (II) and at least one phenol of general formula (III), 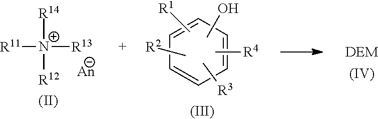
wherein R¹¹ to R¹⁴ are independently selected from linear or branched, substituted or unsubstituted C₁-C₁₈ alkyl, or substituted or unsubstituted C₇-C₂₄ arylalkyl, or
R¹¹ and R¹² together with the nitrogen atom to which they are bonded form a substituted or unsubstituted, saturated or unsaturated 5- to 12-membered cycloalkyl ring that optionally contains 1, 2 or 3 heteroatom(s) selected from O, N, or S as ring member(s); and
An⁻ is selected from chloride, bromide, iodide, fluoride, sulfate, sulfonate, alkylsulfate, or carboxylate, and R¹ to R⁴ as defined above; and
b) reacting at least one substituted 2-chloro-1,3,5-triazine compound of general formula (V) and the deep eutectic mixture obtained according to step (a) to obtain the hydroxyphenyl-1,3,5-triazine compound of general formula (I), 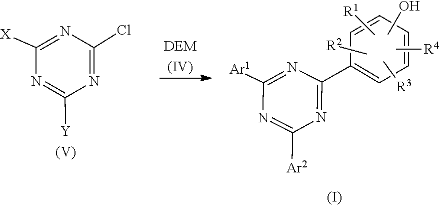
wherein
X is chloro or Ar¹, and Y is chloro or Ar².

26. The process according to claim 25, wherein the molar ratio of the at least one quaternary ammonium salt (II) and the at least one phenol (III) is in the range of 1:1 to 1:15.

27. The process according to claim 25, wherein in step (a) the mixture comprising the at least one quaternary ammonium salt (II) and the at least one phenol (III) is mixed at a temperature in the range of 20 to 150° C. for a time in the range of 5 min to 12 hours.

28. The process according to claim 25, wherein step (a) is carried out without the use of a solvent.

29. The process according to claim 25, wherein the at least one quaternary ammonium salt of formula (II) is selected from choline chloride, choline fluoride, choline bromide, choline iodide, choline acetate, choline salicylate, tetra-n-ethyl ammonium chloride, tetramethylammonium chloride, tetra-n-butylammonium chloride, N,N-diethyl ethanol ammonium chloride, N-benzyl-2-hydroxy-N,N-dimethylethanaminium chloride, N-ethyl-2-hydroxy-N,N-dimethylethanaminium chloride, 2-(chloro carbonyloxy)-N,N,N-trimethylethenaminium chloride, N-methyl-N,N,N-trioctyl ammonium chloride, tris(2-hydroxyethyl)methylammonium methyl sulfate, tributylmethylammonium methyl sulfate or cetethyl morpholinium ethane sulfate.

30. The process according to claim 25, wherein the at least one quaternary ammonium salt of formula (II) is choline chloride.

31. The process according to claim 25, wherein the at least one phenol of formula (III) is selected from substituted or unsubstituted phenol, resorcinol, catechol, quinol, cresol, methoxyphenol, salicylic acid, α-naphthol, or β-naphthol.

32. The process according to claim 25, wherein the deep eutectic mixture (IV) comprises choline chloride, and resorcinol; and
wherein the molar ratio of choline chloride and resorcinol is in the range of 1:1 to 1:15.

33. The process according to claim 25, wherein the substituted 2-chloro-1,3,5-triazine compound of formula (V) is selected from cyanuric chloride, 6-aryl-2,4-dichloro-1,3,5-triazine, or 4,6-diaryl-2-chloro-1,3,5-triazine.

34. The process according to claim 25, wherein the molar ratio of the at least one substituted 2-chloro-1,3,5-triazine compound of formula (V) and the at least one phenol (III) is in the range of 1:2 to 1:20.

35. The process according to claim 25, wherein step (b) involves a step of addition of the at least one substituted 2-chloro-1,3,5-triazine compound of general formula (V) to DES (IV) at a temperature in the range of 30 to 150° C. over a period in the range of 5 min to 12 hours.

36. The process according to claim 25, wherein in step (b), the reaction mixture obtained upon complete addition of the at least one substituted 2-chloro-1,3,5-triazine compound of formula (V) to DES (IV) is heated at a temperature in the range of 30 to 150° C. for 5 min to 600 min.

37. The process according to claim 25, wherein step (b) is carried out without the use of a solvent.

38. The process according to claim 25, wherein the hydroxyphenyl-1,3,5-triazine compound of formula (I) is:

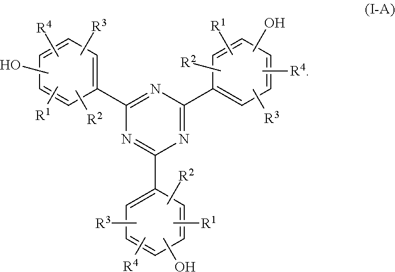

39. The process according to claim 38, wherein the hydroxyphenyl-1,3,5-triazine compound of formula (I) is 2,4,6-tris(2,4-dihydroxyphenyl)-triazine (VI)

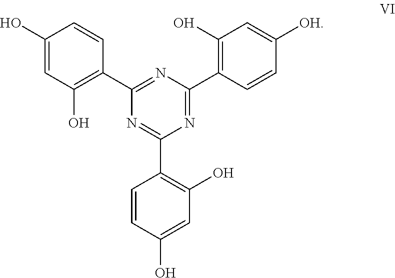 [SMILES: Oc1ccc(-c2nc(-c3ccc(O)cc3O)nc(-c3ccc(O)cc3O)n2)c(O)c1]

40. The process according to claim 25, wherein the hydroxyphenyl-1,3,5-triazine compound of formula (I) is:

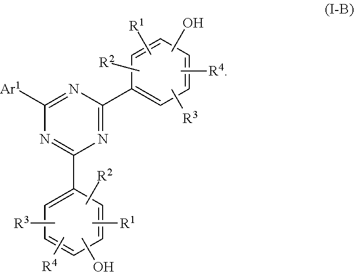

41. The process according to claim 40, wherein the hydroxyphenyl-1,3,5-triazine compound of formula (I) is 2-(4-methoxyphenyl)-4,6-bis-(2,4-dihydroxyphenyl)-1,3,5-triazine (VII).

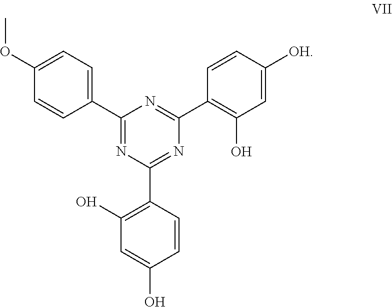 [SMILES: COc1ccc(-c2nc(-c3ccc(O)cc3O)nc(-c3ccc(O)cc3O)n2)cc1]

42. The process according to claim 25, further comprising:
quenching the reaction by adding water to the reaction mixture;
separating the aqueous phase comprising the at least one quaternary ammonium slat of formulation (II) and the at least one phenol of formulation (III); and
recovering the at least one phenol of formula (III) from the aqueous phase.

43. The process according to claim 25, wherein the at least one phenol of formula (III) comprises recovered at least one phenol of formula (III).

44. The process according to claim 25, wherein the yield of the hydroxyphenyl-1,3,5-triazine compound of formula (I) is in the range of 40 to 80 wt,%, and wherein the purity of the hydroxyphenyl-1,3,5-triazine compound of formula (I) is in the range of 80 to 99.9%.
